(19) United States
(12) Patent Application Publication
(10) Pub. No.: US 2010/0255560 A1
(43) Pub. Date: Oct. 7, 2010

(54) METHOD AND APPARATUS FOR CAPTURING VIABLE BIOLOGICAL PARTICLES OVER AN EXTENDED PERIOD OF TIME

(75) Inventors: Charles Call, Albuquerque, NM (US); Trever E.W. Skilnick, Albuquerque, NM (US); Steven T. Strohl, Cedar Crest, NM (US)

Correspondence Address: LAW OFFICES OF RONALD M ANDERSON, 600 108TH AVE, NE, SUITE 507, BELLEVUE, WA, 98004, US

(73) Assignee: MesoSystems Technology, Inc., Albuquerque, NM (US)

(21) Appl. No.: 12/753,768

(22) Filed: Apr. 2, 2010

Related U.S. Application Data
(60) Provisional application No. 61/166,497, filed on Apr. 3, 2009.

(51) Int. Cl. C12N 1/00; C12M 1/12

(52) U.S. Cl. 435/243; 435/307.1

(57) ABSTRACT

Method and apparatus for acquiring a sample of viable biological particles from ambient air, and maintaining the collected viable biological particles at temperature and humidity conditions selected to maintain the viability of the collected viable biological particles. In at least one embodiment, spent collection surfaces are stored in a magazine, and the temperature and humidity conditions in the magazine are controlled. In at least one embodiment, viable biological particles are extracted from ambient air using a particle collector contained in a housing, and the temperature and humidity conditions in the housing are controlled. In at least one embodiment, the temperature and humidity of ambient air entering a particle collector are manipulated to levels selected to maintain the viability of collected particles.

### RELATED APPLICATIONS

This application is based on a prior copending provisional application Ser. No. 61/166,497, filed on Apr. 3, 2009, the benefit of the filing date of which is hereby claimed under 35 U.S.C. §119(e).

### BACKGROUND

Detecting viable biological particles in the outdoor environment, clean or sterilized spaces, hospitals, and other areas is desirable.

Currently, biological particles can be collected by dry filter units. Such units can extract biological particles from the air, but the dry filter substrate tends to desiccate biological particles, such that any sample removed from the dry filter unit will not include a high fraction of viable biological particles (e.g., bacteria and viruses), particularly where such filters are operated over an extended period. Furthermore, it can be difficult to extract a sample of the biological particles from the dry filter.

Agar collection surfaces, such as uncovered Petri dishes, can be employed to collect biological particles; however, such collection surfaces alone are not particularly efficient at extracting biological particles from ambient air. Devices attempting to combine agar with air moving elements, such as Anderson impactors, generally cannot operate for extended periods of time autonomously, and it is somewhat difficult to extract a sample of the biological particles from the agar surface. In addition, because agar is essentially a growth medium, if such samples are stored for any appreciable length of time, the samples will no longer be representative of the biological particles that were present in the sampled air, because of culture growth.

It would be desirable to provide a method and apparatus to efficiently and autonomously collect biological particles over an extended period, without reducing the viability of the collected particles, and which readily facilitates collection of a sample of viable biological particles, sufficiently stabilized, which are suitable for subsequent analysis.

### SUMMARY

This application specifically incorporates by reference the disclosures and drawings of each patent application and issued patent identified anywhere herein.

The concepts disclosed herein involve collecting biological particles while controlling temperature and humidity conditions to maintain a viability of the collected biological particles. Broadly speaking, the concepts disclosed herein encompass three broad temperature/humidity control paradigms: (1) adjust the temperature and humidity of air before depositing particles onto a collection surface; (2) control the temperature and humidity inside the entire particle sampler housing; and (3) control the temperature and humidity inside a volume where spent collection surfaces are stored. Also possible are combinations and permutations thereof. As will be discussed in detail below, a particularly preferred (but not limiting embodiment) combines the control of the temperature and humidity of air entering the sampler with the control of the temperature and or humidity in a volume where collected particles are temporarily stored (under conditions enabling the biological particles to be stored in a viable and non-reproductive state).

In a first aspect of the concepts disclosed herein, a viable biological particle sampler is employed to collect biological particles from ambient air for an extended period of time, where conditions in the biological sampler are configured to support the continued viability of such biological particles. In a particularly preferred embodiment, the temperature and/or humidity of the ambient air is adjusted before depositing particles onto a collection surface. In this preferred embodiment, the temperature and humidity of a volume where collected particles are stored is controlled, until such time as the sample is retrieved from the particle sampler. In at least some embodiments, the particle sampler is capable of automatically changing the collection surface at fixed intervals of time, and moving each collected sample to a compartment or magazine capable of storing a multitude of such samples, within which the humidity and temperature is controlled.

Exemplary characteristics of such a viable biological particle sampler include the ability to collect a concentrated sample of biological particles over an extended period of time (about 12-24 hours or more), while maintaining a viability of the biological particles being collected, means to prevent the desiccation of collected biological particles during extended storage periods (up to 30 days after the sample collection is complete), means to provide a relatively small concentrated liquid sample (about 1-3 ml) for subsequent analysis, and the ability to operate in indoor and outdoor environments, in a variety of temperature and weather conditions.

In an exemplary embodiment, the viable particle sampler includes a housing, a size-selecting inlet, and an air pump in fluid communication with the inlet and an exhaust, the air pump drawing particulate laden air into a hydration tube that couples the inlet to a filter substrate. In an exemplary but not limiting embodiment, the filter substrate is a soluble gel filter such as that sold by SKC, Inc. After passing through/over the gel filter, the air is directed out the exhaust. The viable particle sampler further includes a hydration pump coupled to a water reservoir, to introduce moisture into the hydration tube. In a preferred but not limiting embodiment, the hydration tube is porous, such that water collects in the pores to add moisture to the ambient air introduced into the hydration tube. In another preferred but not limiting embodiment, a solid hydration tube incorporates a water atomizer at its inlet to humidify the air. Sufficient water is introduced into the hydration tube to establish 75-99% humidity conditions (non-condensing) within at the exit of the hydration tube. To allow extended operation, a plurality of filter substrates are included on a carousel driven by a prime mover, such that the filter can be replaced at a predetermined interval or in response to the detection of a predetermined parameter (such as total particle loading), or a user input. An electric heater is included to maintain a temperature of at least 4 degrees Celsius within the air entering the hydration tube, and within the compartment storing the collected samples, allowing extended autonomous operation in freezing cold environments. A cooling system is included to maintain a temperature below about 30 degrees Celsius within the air entering the hydration tube, and at about 4 degrees Celsius in the compartment storing the collected samples, allowing extended autonomous operation in exceptionally hot environments. A controller is logically coupled to the hydration pump, the air pump, the prime mover and filter carousel (if so equipped), a electronic communications interface (if so equipped), the heater, and any temperature, humidity, and other sensors contained within the housing. A battery or power cord is included to provide the required electrical power.

In at least one embodiment, walls of the hydration tube are smooth enough to prevent causing turbulent airflow. In at least one embodiment, walls of the hydration tube are coated with a material to inhibit microbial growth (such as materials provided by Agion, Inc.)

In at least one embodiment for use in relatively colder environments, the heater is configured to heat the incoming air. In at least one embodiment for use in relatively warmer environments, a chiller is configured to cool the incoming air.

In at least one embodiment, the water in the hydration reservoir includes an additive to inhibit the growth of microorganisms in the reservoir. In an exemplary embodiment the additive is sodium hypochlorite. The hypochlorite does not come into contact with the gel filter or biological particles, if the water is evaporating off of the porous walls of the hydration tube. In a related embodiment, the additive is a slowly dissolving solid introduced into the hydration reservoir. In another related embodiment, microorganism growth in the hydration reservoir is inhibited by the use of a continuously or intermittently generated disinfectant such as ozone. A similar continuously or intermittently generated disinfectant system employs salts and an electric current, and is available from Miox, Inc.

In at least one embodiment, such a viable biological particle sampler includes a filter element or size separating elements (noting that virtual impactors can be used as a size selecting element) to ensure that collected particles range in size from about 0.25 μm to about 10 μm, and which specifically excludes relatively larger particles.

In at least one embodiment, such a viable biological particle sampler operates with a flow rate of 20-300 liters per minute. In at least some embodiments, the flow rate is preset, while in other embodiments the flow rate can be adjusted by a user or by an algorithm utilizing real-time measurements from a sensor such as a particle counter or a bioaerosol sensor, such the IBAC™ or AirSentinel™ from ICx Technologies. In still other embodiments, the particle sampler flow rate is set to zero until triggered to turn on by such an algorithm.

In at least one embodiment, such a viable biological particle sampler can maintain internal temperature and humidity conditions proximate to the collected sample that will support the viability of biological particles for a period ranging from about 1 to about 30 days. Alternatively, the sample can be treated or processed such that viability of the biological particles will be supported for periods ranging from about 1 to about 30 days. An example of such a process is lyophilization.

In at least one embodiment, the biological particles are deposited onto a soluble gel filter, and the viable biological particle sampler includes a solvent that dissolves said gel filter to provide a liquid sample for subsequent analysis. Such an embodiment will be able to direct the liquid sample to a sample receptacle or to an integrated analysis unit. A plurality of different gel filters can be provided to provide for extended operation. In at least one such embodiment, a carousal and prime mover are used to replace a used gel filter with a fresh gel filter. Such replacement can be scheduled to occur at predetermined intervals, in response to some detected condition, or in response to a user input. In a related embodiment, the viable particle sampler includes a particle counter or nephalometer to determine when the gel filter is to be changed. In a related embodiment, the viable particle sampler includes a sensor to measure a pressure drop across the filter, to determine if the filter develops a crack or if the filter has become so loaded with particulates that it is necessary to change the filter.

In at least one embodiment, an additive is incorporated into the gel filter to stabilize collected biological organisms. In a related embodiment, an additive that inhibits growth of collected organisms is employed, where such growth/reproduction is inhibited without killing the collected organisms (this technique ensures that the number of biological particles in a sample represents particles removed from ambient air, as opposed to particles/organisms that were not originally present in the ambient air, but which came into existence due to reproduction within the sampler unit).

In at least one embodiment, the gel filter is agitated or heated to aid in dissolution of the gel filter to acquire the liquid sample. In a related embodiment, the solvent includes an additive to facilitate such dissolution. An exemplary solvent is water, and exemplary additives to facilitate such dissolution include salts and ethanol. In a related embodiment, the solvent includes an additive or buffer to control a pH of the resulting solution, such that the resulting solution (including a sample of the viable biological particles) does not reduce a viability of the biological particles.

In at least one embodiment, the viable biological sampler includes means to reduce the volume of water that needs to be stored in the hydration reservoir. In one such embodiment, moisture is scavenged from air after being filtered by the gel filter and before it is exhausted from the housing. Desiccant technology is regularly used to scavenge moisture from air. Refrigeration/chillers and dehumidifiers can also be used to scavenge water from the exhaust. Such techniques will require additional amounts of electrical power, but in certain environments power is more easily (or more cost-effectively) supplied than water.

In at least one embodiment, the viable biological sampler includes means to rehydrate the filter substrate or gel filter periodically, such that it can operate for long periods of time without suffering degraded performance. In an exemplary but not limited embodiment, water is misted into the hydration tube to add moisture to the incoming air (as opposed to simply wetting the walls of the hydration tube). In another embodiment, water (or a mixture of water and additives to enhance viability) is misted directly onto the filter substrate (note that if a water soluble gel filter is used for the filter substrate, care must be taken not to use so much water that the gel filter dissolves before a liquid sample is desired).

In at least one embodiment, fresh filters are stored in a first stacked filter magazine, and used filters are stored in a second stacked filter magazine, and a carousel moves filters between the first and second stacked magazines. In a preferred but not limiting embodiment, the fresh magazine is refrigerated to prevent microbial growth and desiccation on the fresh filters. In a similarly preferred but not limiting embodiment, temperature and humidity conditions in the spent filter magazine are maintained at levels selected to maintain high viability of the collected biological particles.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is a block diagram schematically illustrating the basic functional elements of an exemplary viable biological particle sampler implementing the concepts disclosed herein;

FIG. 2 schematically illustrates a portable air sampler implementing the elements of FIG. 1;

FIG. 3 is a more detailed functional diagram schematically illustrating functional elements of another exemplary viable biological particle sampler implementing the concepts disclosed herein;

FIG. 4 schematically illustrates a carousel including a plurality of collection surfaces to enable extended operations;

FIG. 5 is a functional diagram schematically illustrating functional elements of another exemplary viable biological particle sampler implementing the concepts disclosed herein, which includes magazines enabling a plurality of collections surfaces to be employed, to enable extended sampling; and

FIGS. 6.1-6.8 schematically illustrate an exemplary filter changing paradigm for use with the sampler of FIG. 4.

### DESCRIPTION

### Figures and Disclosed Embodiments Are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. Further, it should be understood that any feature of one embodiment disclosed herein can be combined with one or more features of any other embodiment that is disclosed, unless otherwise indicated.

The term viable biological particle is used in the specification and claims that follow. It should be understood that this term encompasses living microorganisms, including but not limited to bacteria, viruses, fungi, archaea, protists, microscopic plants, and microscopic animals. While some of ordinary skill in the art do not consider viruses to be alive, in the context of the specification and claims that follow, viruses that have not been deactivated (i.e., that are capable of causing infection) are to be considered to be encompassed by the term viable biological particle.

The following description first describes an embodiment for an exemplary particle sampler in terms of its basic functional elements. Then, each functional element is discussed in greater detail, and finally, additional exemplary embodiments are discussed.

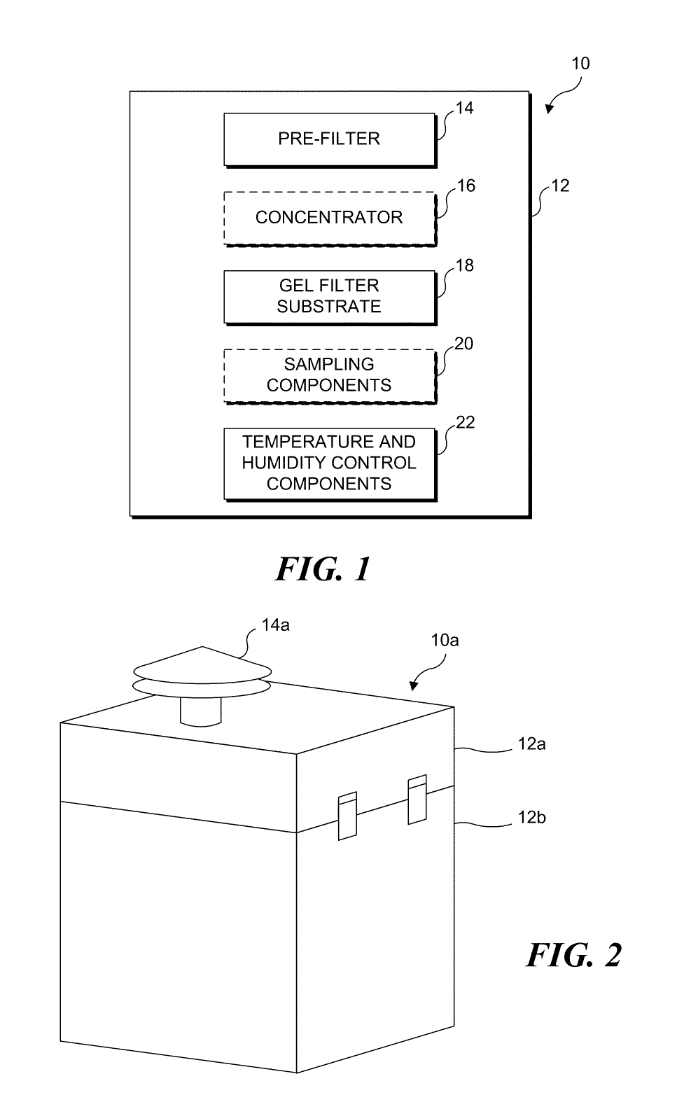

FIG. 1 is a functional block diagram schematically illustrating the basic functional elements of an exemplary viable biological particle sampler 10, which includes a housing 12, a pre-filter 14 (which in some embodiments may partially extend beyond the housing), an optional concentrator 16, a particle collector 18 (preferably implemented using a gel filter, although other substrates could be employed), an optional sampling component 20, and temperature and humidity control components 22. Commercial embodiments of sampler 10 are likely to be available with and without the analytical component(s), such that customers already having analytical components (such as chemical sensors, biological sensors, or other analytical instruments) can use the air sampler with the equipment that they already have, or samples can be removed from sampler 10 for analysis externally of the sampler.

Housing 12 is implemented to protect the additional functional elements discussed below, to facilitate transportation of the air sampler, and to define one or more volumes in which temperature and humidity conditions can be controlled. Those of ordinary skill in the art will readily recognize that air samplers of many different form factors and sizes can be implemented consistent with sampler 10. Relatively larger air samplers capable of sampling relatively larger volumes (or volumetric flows) of air may be implemented in buildings, whereas relatively smaller air samplers can be made to be portable, so as to be readily moved from one location to the next to sample air (or other fluid) in a plurality of different locations.

Pre-filter 14 is a device that performs one or more of the following functions: (a) removes over-sized particles that are too large to be of interest (for example, those greater than 10 microns in diameter), (b) rejects or removes rain, snow, and other water precipitation, (c) restricts insects from crawling or flying into the apparatus, and (d) rejects or removes other flying debris. Pre-filters can be implemented using inertial impactors configured to remove such oversized particles (the term oversize indicating that the particles are larger than a particle size that is of particular interest), virtual impactors, or filters including a plurality of pores smaller in size than the oversized particles.

Optional concentrator 16 is configured to discard a portion of the air introduced into the concentrator without also discarding a majority of the particles of interest, thereby increasing the concentration of particles of interest in the remaining portion of the air (i.e., that portion of the air that has not been discarded) to enhance collection efficiency. Virtual impactors represent a particularly preferred technology used to implement such concentrators. A virtual impactor is a device that will separate a fluid flow (such as air) into a minor flow (i.e., a smaller fraction of the fluid flow) containing a majority of particles larger than a cut size, and a major flow (i.e., a major fraction of the fluid flow) containing particles smaller than the cut size. Virtual impactors are available that exhibit relatively low pressure drops (which may be desirable because relatively low pressure drops minimize power requirements) across each stage and that can be injection molded at a relatively low cost. For example, particles ranging from about 0.25 to about 10 μm in size can be concentrated, and the volumetric flow of air significantly reduced. Multiple virtual impactors can be arranged in series to achieve higher particle concentrations.

Particle collector 18 is configured to collect particles of interest from the remaining portion of air (i.e., that portion of the air that has not been discarded by the concentrator). In general, particle collector 18 is a collection surface that removes particles from the air by impaction (i.e., the particles entrained in the air collide with the collection surface and are retained thereon, or air into which such particles are entrained passes through a porous collection surface). As discussed in greater detail below, an exemplary collection surface is a gel filter.

Optional sampling component 20 is configured to obtain a sample from the particles deposited upon the collection surface, and to prepare the particles for analysis by an analytical component. The type of sample obtained and the sample preparation required will vary depending on the specific analytical component employed. For example, some analytical components require dry samples, some require wet samples (i.e., samples contained in a volume of liquid), and still other types of analytical components require gaseous or vaporous samples. Gaseous and vaporous samples can be obtained by desorbing a sample from a surface using heat (which can be supplied by various elements, such as an infrared lamp, an electrical resistive heater, or a laser). Gaseous/vaporous samples can also be obtained by dissolving the sample in a solvent and flash vaporizing the solvent.

In a particularly preferred, but not limiting group of embodiments, sampling component 20 is configured to obtain a liquid sample from the particles deposited upon the collection surface. In some embodiments, biological particles can be rinsed off of the collection surface using a liquid. Where the collection surface is a gel filter, sampling component 20 preferably generates a liquid sample by dissolving the gel filter using a solvent (an exemplary but not limiting solvent being water).

In at least one embodiment, described in greater detail below, sampling component 20 employs the detection of stimulated fluorescence to verify that biological particles are present on a collection surface. That verification can be the end of the analytical process, or can be used to determine which of a plurality of different collection surfaces should be processed to obtain a liquid sample (in at least some embodiments, where no biological particles are indicated by the fluorescent analysis, no liquid sample need be obtained from that particular collection surface, thus minimizing the amount of liquid samples that need to be generated).

In some embodiments, a portion of the collection surface may be removed to obtain a sample, and another portion of the collection surface is placed in fluid communication with the particulate laden air to collect additional particles to be used to obtain a future sample. In still other embodiments, a mechanism is included to clean the collection surface after a sample has been collected. Such cleaning mechanisms include, but are not limited to, liquids, compressed air, cleaning pads, and cleaning brushes.

As noted above, in some embodiments where on-board analysis is desirable, sampling component 20 can include analytical components, which can be implemented using various types of analytical instruments, including but not limited to: fluorescence-based sensors; chemical sensors; particle counters; spectrophotometers; gas chromatographs (GC); mass spectrographs (MS); and combinations thereof (for example, a GC/MS). Clearly, the sampling component implemented is based on the analytical component that will be employed.

Not specifically shown in FIG. 1 are additional components, such as tubing and hoses used to move air from one portion of the sampler to another, an inlet to introduce air into the air sampler, one or more exhaust ports to discard air, fans or pumps to move air through the air sampler, a battery or other electrical power source (or an electrical conductor configured to bring electrical power into the air sampler from an outside source, such as line voltage) to energize components requiring electrical power, a data port configured to enable analytical data to be output from the analytical component, and a controller configured to control overall operation of the air sampler. FIG. 1 is provided to highlight the most important functional elements of air sampler 10, and those of ordinary skill in the art will readily recognize that the elements noted above as not shown will likely be incorporated into the air sampler to enhance its functionality or utility. Additional Figures discussed in detail below provide additional information relative to some of these other components.

Several factors, beyond the type of sampling or analytical component that will be used to analyze the sample obtained by air sampler 10, can affect the specific implementation employed. For example, the flow rate of the sampler is dependent upon the power requirement and size requirement of the sampler. Thus, air samplers intended to have higher flow rates will generally be larger and require more power. It should also be recognized that for viable biological particle samplers intended to operate for an extended period of time, a significant amount of mass may need to be dedicated to water storage to maintain desired humidity conditions in the sampler. Such water storage is part of temperature and humidity control components 22, and will be discussed in detail below.

In determining a design for a sampler, each of the following can represent an important consideration: environmental compatibility, sensor system compatibility, concentration factor, particle size selectivity, reliability, logistics (size, weight, power, and noise), operating cost, and initial cost. Different end-users having different applications in mind will weigh these factors accordingly, based on their requirements. Some trade-off between these parameters might be employed to customize a sampler design to the specific requirements of a user.

With respect to environmental compatibility, relevant factors associated with outdoor environments include operating humidity, temperature, susceptibility to wind, rain, and the presence of pollutants in the environment (such as engine exhaust and other pollutants in an urban environment). Relevant factors associated with indoor environments can include: low humidity (often associated with mail rooms), a high loading of paper dust (also often associated with mail rooms), and a high loading of other particulate contaminants (often associated with battlefields and subways).

In one embodiment, schematically illustrated in FIG. 2, a portable sampler 10a including an integrated inlet/pre-filter/concentrator 14a is implemented by coupling an omni-directional inlet with a virtual impactor configured to separate large particles from small particles. Note a portion of inlet 14a extends beyond housing 12. The minor flow from the virtual impactor/concentrator is then directed to an impaction surface to collect biological particles. Sampler 10a can include a light-weight/impact resistant two-piece housing, with an upper portion 12a and a lower portion 12b, configured to enable easy access to internal components for maintenance and replenishment of consumables (such as liquids and filter media/collection surface).

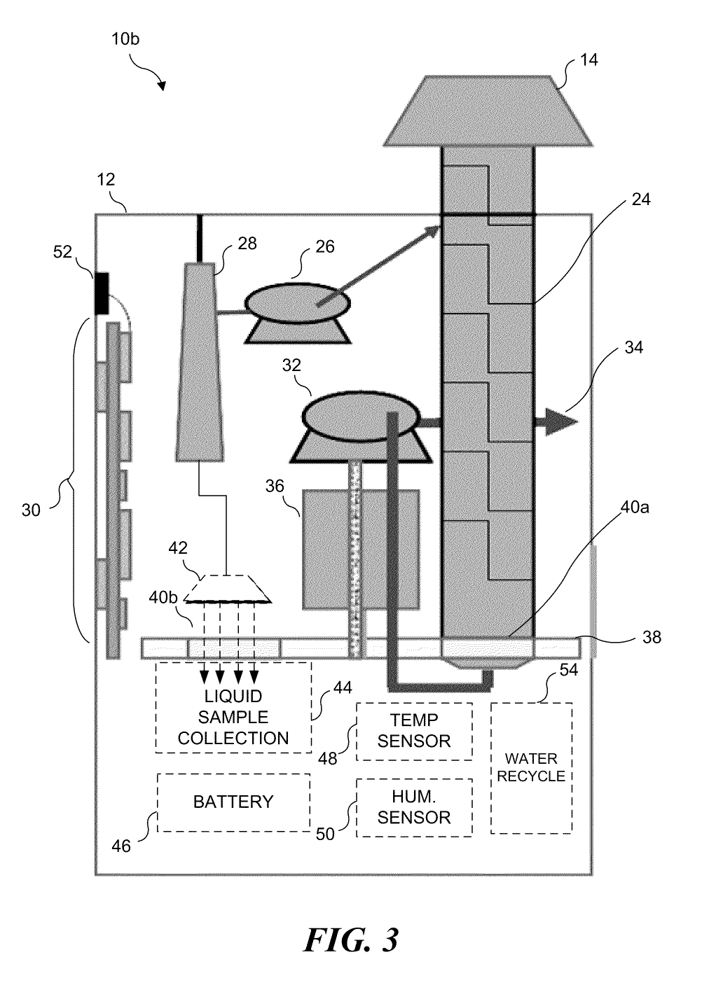

FIG. 3 is a particularly preferred (but not limiting) embodiment of a viable biological particle sampler in accord with the concepts disclosed herein, including a plurality of gel filters to enable extended operations. Note that sampler 10b of FIG. 3 is more detailed, and illustrates an exemplary (but not limiting) configuration of a collection surface handling component that enables a plurality of collection surfaces to be used to enable extended operations. Sampler 10b includes housing 12, omni-directional sizing inlet 14, a hydration tube 24 (representing a volume coupling the inlet in fluid communication with the collection surface, the sampler being configured to control at least one of a temperature and a humidity in such a volume), a first gel filter 40a (in fluid communication with the inlet, such that particles entrained in air entering the inlet are deposited thereon), a second gel filter 40b (to be placed in fluid communication with the inlet once first gel filter 40a is saturated, as indicated by a pressure drop of a predetermined value, or after a predetermined period of time has elapsed), a carousel 38 supporting the first and second gel filters (noting that in some embodiments carousel 38 may support more than two gel filters), a prime mover 36 (an exemplary but not limiting prime mover being an electric motor) for rotating the carousel, an air pump 32 for drawing air into the housing via the inlet, and causing particle laden air to impact on the gel filter in fluid communication with hydration tube 24, an exhaust port 34 enabling filtered air to escape from the housing, a hydration pump 26 in fluid communication with a hydration reservoir 28 and hydration tube 24, a printed circuit board based controller 30, and a data port 52 (such as a USB port, a serial port, a parallel port, an IR port, or a wireless data port) logically coupled to the controller. The controller can be implemented as a hardware controller (such as an application-specific integrated circuit) or as a software-based controller (i.e., a computing device including a processor that executes machine instructions stored in a memory to carry out control functions). Not specifically shown are wires and tubing coupling the various components together in a generally conventional manner. Optional additional components include a battery 46 (in case line voltage or external power sources are not employed or available), one or more temperature sensors 48 (logically coupled to the controller), one or more humidity sensors 50 (also logically coupled to the controller), a nozzle 42 for directing water from hydration reservoir onto a gel filter that has been used to collect biological particles, a liquid sample collection reservoir 44 configured to collect a liquid sample of collected biological particles (such particles having been rinsed off of the collection surface, or having been deposited upon a gel filter that was dissolved), and a water recycling component 54 (to extract water from air after particles have been removed, and before such air is exhausted, to reduced an amount of water used by the device; the recycled water being filtered and added to the hydration reservoir). It should be recognized that the relative locations of the sensors are not intended to represent a preferred location, as the sensors will be positioned to collect data regarding specific locations where temperature and humidity control is desired.

Note that gel filter 40a in FIG. 3 can be considered to be positioned at a sampling location or sampling position, as it is in fluid communication with hydration tube 24, and thus is able to collect biological particles entrained in the air introduced into the hydration tube.

Not shown in FIG. 3 is a temperature control component (beyond the temperature sensors). In an exemplary and not limiting embodiment, heating and cooling devices (such as electric heaters and Peltier coolers, noting that such elements are exemplary and not limiting) are included in one or more locations in the device, to enable the temperature to be controlled to a level selected to maintain a viability of collected biological particles. In at least one embodiment, the temperature in the volume defined by hydration tube 24 can be controlled. In a preferred but not limiting embodiment, an electric tape heater is coiled around an external surface of hydration tube 24.

As discussed above, hydration tube 24 is, in an exemplary but not limiting embodiment, implemented using a porous material, such that water from hydration reservoir 28 is used to wet the walls of the porous tube, so that moisture is added to dry ambient air entering the hydration tube. In cases where the ambient air is already moist, it may be desirable to actually remove moisture from the ambient air. If this is required, humidification control equipment (such as desiccators), can be employed to dry the ambient air to a desired degree. Placing a humidity sensor in fluid communication with the hydration tube (or in the ambient air) will provide data that can be used to determine what humidity conditions should be established in the hydration tube.

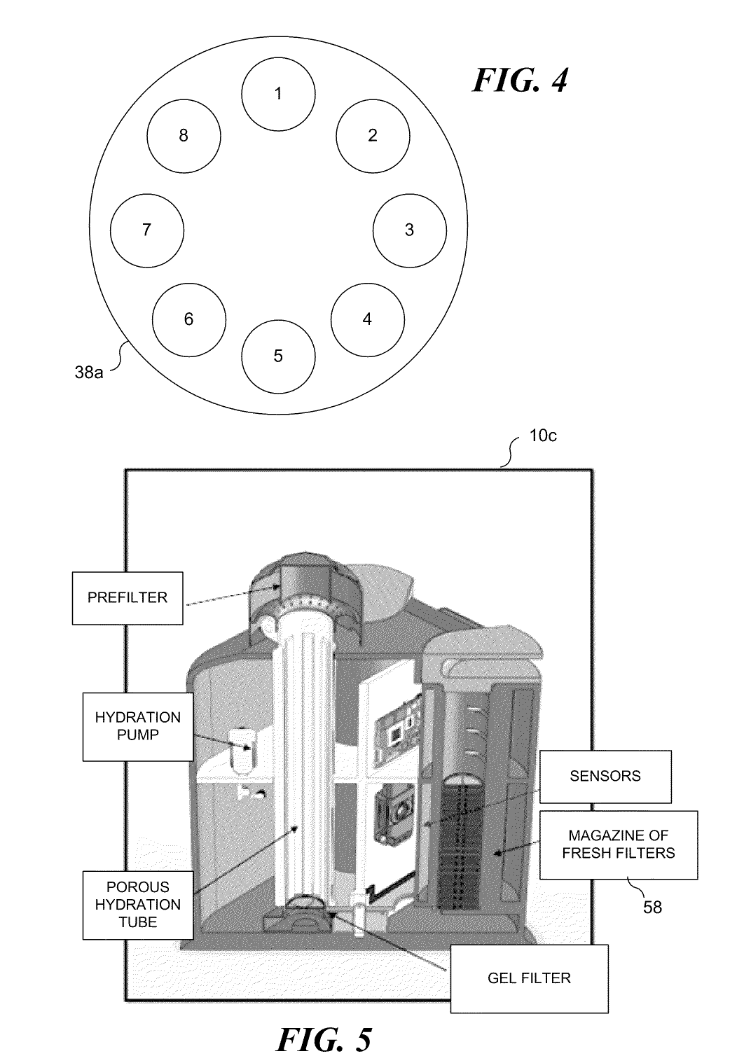

FIG. 4 schematically illustrates an exemplary carousel 38a for use with the viable biological particle sampler of FIG. 3. Carousel 38a is of sufficient size to accommodate 8 different gel filters. It should be understood that the dimensions of the housing, the diameter of the carousel, and the sizes of the gel filters will determine how many different gel filters can be accommodated by a carousel, thus 8 gel filters is exemplary, and not limiting. In other embodiments, magazines for storing additional gel filters can be included, enabling devices with even more gel filters to be fielded.

FIG. 5 is a block diagram schematically illustrating functional elements of another exemplary viable biological particle sampler, implementing both a fresh gel filter magazine and a spent gel filter magazine, and a filter changing component. Note that sampler 10c of FIG. 5 is based on sampler 10b of FIG. 3 (thus, components from the sampler of FIG. 3 that are not specifically shown in FIG. 5 can be incorporated into sampler 10c), but has been modified to include a fresh filter magazine 58, and a spent filter magazine (not visible in the cut-away view of FIG. 5). The filter magazines enable sampler 10c to be deployed for longer periods of time than the sampler of FIG. 3, because the magazines provide more storage for gel filters. In an exemplary but not limiting embodiment, not only are the temperature and humidity controlled in the hydration tube, but also in the storage magazine for the spent gel filters. Particularly where gel filters are replaced frequently, if desired one or both of the temperature and humidity control of the hydration tube can be eliminated, in favor of providing such controls in the spent filter storage magazine. In at least some embodiments, the temperature and/or humidity control will be provided in the hydration tube, the spent filter magazine, and the fresh filter magazine. It should also be recognized that it may be desirable to establish different environmental conditions in different volumes. Thus, the concepts herein encompass embodiments wherein the humidity and temperature conditions in the spent filter magazine, the hydration tube, and the fresh filter magazine are different. If it is established that the biological samples collected may include biological organisms having different environmental requirements, a plurality of different spent filter magazines can be provided, each having different environmental conditions provided therein, optimized for maintaining the viability of different biological organisms. In at least one embodiment, an additional hydration tube and sampling position can be provided, where the environmental conditions for the additional hydration tube/sampling position are also separating controlled.

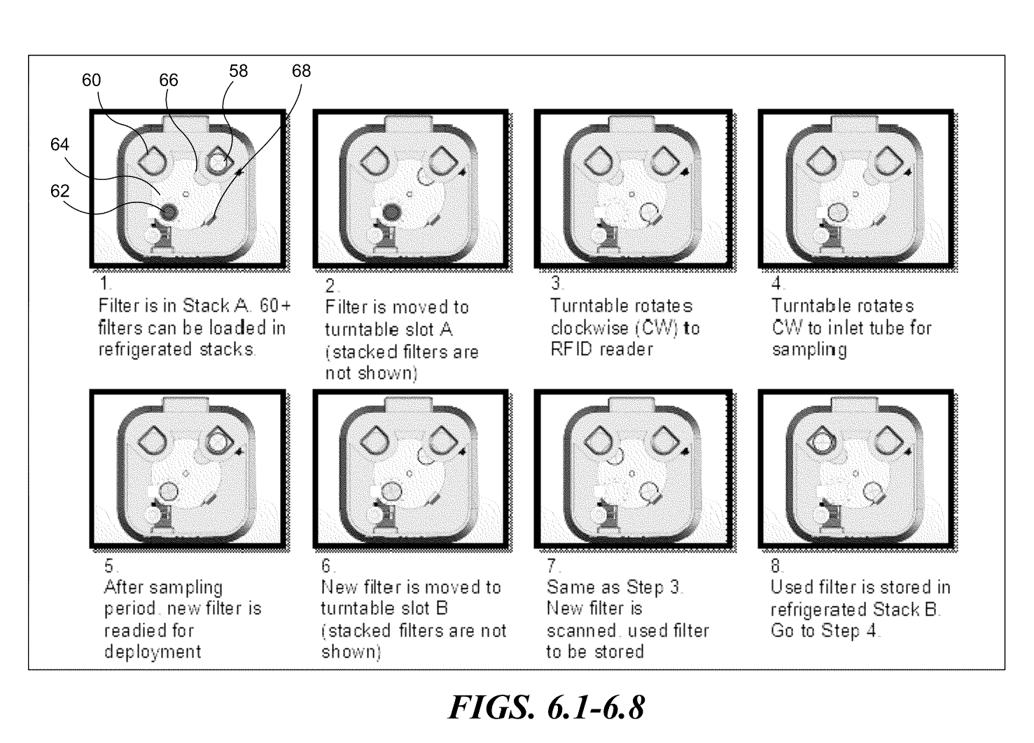

Referring to FIG. 5, sampler 10c requires a filter transfer mechanism to move gel filters (or other collection surfaces) between the fresh filter magazine, the sampling position, and the spent filter magazine. FIGS. 6.1-6.8 schematically illustrate the operation of an exemplary carousel/turntable based transfer mechanism. Elements of this exemplary system are specifically numbered in FIG. 6.1, but such numerals have been omitted from FIGS. 6.2-6.8 for simplicity. While the elements undergo state changes in the various Figures, it should be recognized that the elements remain the same throughout the Figures. It should also be recognized that while the exemplary system includes only a single spent filter magazine, the system can be readily modified to accommodate a plurality of spent filter magazines.

Referring to FIG. 6.1, the exemplary filter transfer mechanism includes fresh filter magazine 58, a spent filter magazine 60, and a carousel 64 including a pair of filter slots 66. A first filter slot is visible proximate fresh filter magazine 58, and a second filter slot is diametrically opposed on the carousel, and is obscured in FIG. 6.1 by a sampling position 62. A filter in sampling position 62 is in fluid communication with the hydration tube shown in FIG. 5, and is thus exposed to the particulate laden air. In an exemplary but not limiting embodiment, filter transfer mechanism includes an RFID tag reader 68, to read RFID tags incorporated in each filter, as the filters are moved between the fresh filter magazine and the sampling position. It should be understood that the RFID technology is not required, but its incorporation into the device will enable data collected by sensors and the processor (generally discussed above) to be readily matched to individual filters.

In FIG. 6.1, no filter has yet been used to collect any biological particles, and carousel 64 has been rotated such that one of the filter slots is positioned to receive a fresh filter from fresh filter magazine 58. As illustrated in FIG. 5, the filter magazines store the filters in a stack. In an exemplary but not limiting embodiment, the filter magazines hold 60 filters.

In FIG. 6.2, a filter is being loaded into the filter slot positioned next to fresh filter magazine 58. In FIG. 6.3, the carousel/turntable rotates about one quarter of a revolution to bring the fresh filter just loaded into the filter slot adjacent to RFID tag reader 68. In FIG. 6.4, the loaded carousel/turntable rotates about one quarter of a revolution to bring the filter loaded in FIG. 6.2 and read in FIG. 6.3 to the sampling position, where biological particles will be deposited as the air pump discussed above draws air into the sampler. The sampler will be operated until the filter has collected a sufficient sample. In some embodiments, the sampling time is preset, and spent filters will be replaced with fresh filters according to a predetermined schedule (in an exemplary, but not limiting embodiment, filters are replaced every two hours over a 5 day period). In other embodiments, a pressure sensor is used to determine a pressure drop across the filter, and when the pressure drop increases to a predetermined level (indicating the filter is saturated), the spent filter is replaced. The pressure sensor can also be used to detect a broken or cracked filter (the pressure drop decreases, indicating a loss of integrity), and under such circumstances the filter can also be replaced. A particle counter can also be used to determine particle loading indicative of the filter replacement frequency. Note that in FIG. 6.4, the rotation of the carousel has placed the second filter slot next to the fresh filter magazine.

In FIG. 6.5, magazine 58 is being readied to deploy another fresh filter (this Figure ignores the additional fresh filters that would be stacked above the fresh filter that will be deployed). In FIG. 6.6, the new filter is being loaded into the filter slot positioned next to fresh filter magazine 58. In FIG. 6.7, the carousel/turntable rotates about one quarter of a revolution to bring the fresh filter just loaded to RFID tag reader 68, and the spent filter that has been used to collect particles to spent filter magazine 60. In FIG. 6.8, the spent filter is stored in the spent filter magazine.

Additional embodiments are discussed below.

In at least one embodiment, the viable biological particle sampler is based on gelatin filter technology. In an exemplary, but not limiting embodiment, the gel filter is exposed prior to, during, and after sample collection to temperature and humidity conditions selected to maintain the integrity of the gel filter and to optimize the viability of the biological material captured on the filter. The viable biological particle sampler is designed to collect air samples and condition the samples through particulate filtration and temperature and humidity adjustment prior to delivery of the airstream to the gel filter. An automated gel filter transfer system inserts fresh filters into the sample stream and then moves used filters to a storage area that supports viable storage of samples. A fresh filter is inserted into the collection stream and the process starts over, providing continuous collection.

Exemplary, but not limiting subsystems include: (1) an omni-directional pre-filter for removal of large particles prior to collection; (2) an inlet tube for conditioning of the incoming air temperature and humidity to maintain appropriate conditions for the gel filter and organism viability; (3) a collection filter for collection of bio-aerosols at high efficiency; (4) a sample handling and a mechanical storage system for moving filters from a supply magazine to a sampling station, and then to a storage magazine, and (5) components for maintaining appropriate environmental conditions in each area (such environmental conditions having been selected to maintain a viability of the biological samples).

Gel filters offer several advantages over inertia-based samples for the collection of small biological components from the air. These include: (1) a high collection efficiency even of submicron particles, which is very difficult to accomplish with inertia-based approaches such as impactors, impingers or cyclones; (2) a demonstrated ability to maintain viability of particles over several hours of collection time (inertial-based particle collectors or high voltage-based approaches, such as electrostatic precipitators, significantly reduce the viability of some bio-aerosols during the collection process); and (3) the ability to process in excess of 100 liters per minute (lpm) with a single filter.

For collection of bio-aerosols, gel filter materials are designed to be compatible with standard laboratory analytical techniques, and have been used extensively for microbial analysis through culturing, immunoassay, and PCR. A primary drawback of using gel filters is their performance in extreme temperatures and humidity levels. Thus, the viable biological particle samplers disclosed herein that employ such gel filters maintain an environment that will both maintain the viability of the biological particles, as well as maintain an environment suitable for using gel filters. A hydrated and heated inlet tube can be used to maintain the temperature and relative humidity at a suitable range for efficient functioning of the gel filter and maximum viability of the organisms over the sampling period. These gel filters are not appropriate for use in freezing conditions; therefore, the incoming air will be heated prior to reaching the filter, as necessary. Humidity control via a wetted tube wall will also be used to increase the humidity, again as needed. Sensors will monitor the ambient conditions to allow for active control of the temperature and humidity during sampling.

Design studies have indicated that the volume of hydration water required for the worst-case environment will weigh approximately 50 pounds, for an embodiment designed to operate continuously for 30 days. However, in less demanding environmental conditions, the water requirement will drop significantly (most environments should require <25 pounds of water for the same duration). The collector hardware for such an embodiment is expected to weigh about 15 pounds. Weight and mass reductions can be achieved where a source of water exists, or where a water recovery system is employed. High particulate loading may present a challenge for the gel filters due to the increase in pressure drop, and more than one filter may be required in a 24-hour period when operating in highly loaded environments. Thus, the incorporation of a plurality of filters in a carousel, or the use of the stacked magazines will enable longer duration employment.

In an exemplary but not limiting embodiment the controller will be implemented using a printed circuit board, with sufficient memory resources to hold 30 days of data. Exemplary (but not limiting) data to be recorded can include: (1) the location and serial number of the unit; (2) temperature and humidity data for samples and filters stored in the filter magazines; (3) fault sensor data; (4) RFID tag numbers associated with each collected sample (note that to facilitate tracking of samples, each different collection surface can incorporate a unique RFID tag); (5) RFID tag numbers of the unused filters contained in the fresh filter magazine; (6) all data associated with each RFID tag for which a sample was collected, including the starting and ending time and date of the collection period; (7) the RFID tag numbers of each unused filter contained in the fresh collection surface/fresh filter magazine; and (8) data corresponding to the ambient temperature and relative humidity conditions during sample collection.

For units designed to operate on line voltage or external power sources, the unit can be designed with a limited amount of battery back-up power if needed. In an exemplary embodiment, the sampler is designed such that regardless if power is lost, stored data and accurate time stamp information will not be lost, and the unit will come back on line when power is restored.

Exemplary viable biological particle samplers will be designed to be maintainable in the field, with field-replaceable subsystem modules, allowing mean-time-to-repair of less than 30 minutes for any subsystem failure. Fault sensors will be capable of determining which subsystem generated the fault, and what components are in need of repair, calibration, or replacement.

As indicated above, at least one embodiment of the viable biological particle sampler will incorporate filters that include an RFID tag that automatically stores information related to the sample, including a unique identification number, the location and serial number of the viable biological particle sampler in which the sample was collected, the starting and ending time and date of each aerosol sample, information regarding the range of ambient temperature and relative humidity during the sample collection, and other appropriate information. Preferably, the information will be maintained in a nonvolatile form that cannot be overwritten. Retrieval of information at a laboratory analyzing the sample will provide critical information in the event that a positive result is obtained.

With respect to the gel filters, relatively larger gel filters (available from Sartorius (or SKC)) can be used with a relatively lower flow rate, to provide additional surface area to reduce a pressure drop needed to pull air through the filter. Empirical data indicate that such gel filters are essentially 100% efficient at collection of single spores and cells, and can also be successfully used for viral sampling, particularly for MS-2 viruses.

In operation, an exemplary gel filter based viable biological particle sampler will use a pump to pull air through an omni-directional inlet pre-filter into a sampling tube designed to control the temperature and humidity of the sampled air. Commercially available gel filters are suitable for use in air temperatures as high as 30° C. In a high humidity environment with lower temperature, the air will be heated to as high as 30° C. to reduce the relative humidity to within a range of 85-95%. For low humidity environments, the humidity will be increased by controlling the wetness and temperature of the sampling tube wall. At 20 liters per minute with a 2-cm tube diameter, the velocity in the tube is about 106 cm/s. A 30 cm tube will have a residence time of about 0.28 seconds. Empirical data indicates that such parameters should result in laminar flow. Under these conditions, to establish a uniform temperature and humidity across the flow, the required tube length is nominally 30 cm. The sampling tube itself is preferably implemented using a hydrophilic micro-porous material, which will be maintained in a wetted condition via the water pump and de-ionized water from the hydration reservoir. A microbial growth inhibitor such as calcium hypochlorite can be added to the water to inhibit microbial growth within the wetted porous sampling tube. In an exemplary but not limiting embodiment the volume of air sampled in a 24-hour period is about 28.8 cubic meters. Very dry cold air will hold approximately 2 gm water per kilogram of air. Saturated air at 30° C. will hold about 28 g/kg, and, thus, the system needs to be able to supply up to 26 g/kg, which is equivalent to 900 ml/day as a worst case scenario. A 30-day operating life would then require approximately 25 liters of water, which is slightly less than one cubic foot of water. Most environments will require 3-5 times less water, and water requirements over a 30-day period at a particular location can likely be predicted to within 20% using historical data. In addition, sampling may be possible at 4° C. in cold environments, dramatically lowering the water requirement.

In an exemplary gel filter based viable biological particle sampler including a plurality of gel filters, the gel filters will be stored prior to and after sampling. For storage after sampling, in an initial empirical embodiment, the filters will be stored at a temperature of about 4° C., unless testing indicates that a higher temperature is required to maintain viability of the collected biological material. This relatively low temperature will significantly reduce reproductions rates (such that a relative number of biological particles in the samples will remain representative of the relative numbers of biological particles present at the time the sample was collected). Storage of the filters prior to sampling can accommodate a wider range of conditions, nominally 4-30° C. Exemplary heaters include tape heaters and Peltier coolers. Consumables include the gel filters, hydration water, and power. A worst case cooling load would be from 51° C. to 30° C., or a change of 21° C. For a 20-lpm flow and maximum temperature difference, this equates to about 8.4 watts of cooling power, which is readily achievable. For cold environments, the heating requirement is approximately 10 watts, which is also quite manageable. Since the storage areas for the filters will not have the constant air flow present in the hydration tube, the conditions in the filter storage areas can maintained with relatively little power expenditure, once the desired temperatures have been achieved.

For embodiments intended to be deployed long term, a key feature is the autonomous filter change system, to support long-term threat monitoring. The fresh filter magazine stores fresh filters that can be inserted into the sample path for sample collection, and then transferred to a refrigerated storage magazine to ensure sample viability. In at least one embodiment, the automated mechanism will be used to move filters between a fresh filter magazine, a sampling position, and a spent filter magazine. Then the automated mechanism will be used to stack spent/exposed filters under refrigerated conditions until such spent filters are retrieved for analysis.

In at least some embodiments, a particle counter will be included to monitor the ambient conditions. Data from the particle counter can be used to determine how frequently the filter media should be changed (relatively higher levels of ambient particles will require more frequent filter changes, while relatively lower levels of ambient particles will require less frequent filter changes).

In at least some embodiments, liquid sampling components will be integrated into the viable biological particle sampler to enable liquid samples to be automatically collected. For extraction of the sample into liquid, the gel filter is placed into a suitable container and liquid solvent added to dissolve the filter, or while in the sampling position, the filter is exposed to solvent and allowed to dissolve into a container disposed below. Exemplary solvents include water, sterile water, saline, and peptone water, which preferably reduce osmotic shock and promote the viability of the collected organisms. Empirical studies indicate that commercially available gel filters can be dissolved in as little as 3-5 ml of water based solvent. Heating the solvent liquid to 35-40° C. facilitates dissolving of the gel.

As discussed above, exemplary embodiments of the viable biological particle sampler include a hydration tube coupled in fluid communication with the inlet enabling air to be introduced into the device, and the collecting surface. In an exemplary, but not limiting embodiment, a tape heater and a cooling coil are wrapped around the exterior of the hydration tube, enabling control of temperature conditions in the hydration tube. Because of the wide range of temperatures that ambient air can exhibit, the temperature conditioning elements preferably enable the hydration tube to be heated or chilled, depending on the temperature of the ambient air. It should be understood where ambient air is hot and humid, the incoming air may need to be dehumidified, and the condensate can be captured and reused if desired.

In a preferred but not limiting embodiment of the viable biological particle sampler, a seal removably couples a distal end of the hydration/inlet tube to the collection surface/filter.

In a preferred but not limiting embodiment of the viable biological particle sampler, a plurality of sensors are included in the sample, to enable conditions within the sampler to be monitored. Exemplary sensors include but are not limited to internal sensors for airflow rate, water flow rate, pressure drop across the filter, and temperature and humidity measurements. Additional sensors can include Hall sensors to monitor electrical current to pumps and motors. Data from the Hall sensors can be used diagnostically, to determine if the pumps and motors are operating properly (i.e., are not burned out, etc.). Such diagnostic data can be used to verify normal operation and to spot component failures. In a preferred but not limiting embodiment of the viable biological particle sampler, a printed circuit board type controller will be logically coupled to the sensors, the pumps, and temperature and humidity control components. Preferably, such a controller board will include a communication port (wireless or hard wired) to enable data and programming changes to be communicated to a remote computing device (such as a desktop or laptop computer). Communication with a remote computer will allow data logging and remote analysis of fault codes.

In at least one embodiment, a fluorescence-based analytical component will be included. Biological material often includes proteins and molecular components that emit characteristic fluorescence when stimulated by light of the appropriate wavelength. An exemplary fluorescence based analytical component includes a light source (such as a laser or laser diode) emitting the required stimulating waveband, and a photo detector capable of collecting the characteristic fluorescence. Such a fluorescence based analytical component can be configured to collect fluorescence from filters after they have been exposed (i.e., while the collected biological particles are disposed on the filter surface), or from a liquid sample generated by dissolving an exposed filter. emission.

It should be noted that empirical devices will be designed and fabricated to prevent or reduce the following potential failure modes. Gel filters may crack or fail due to brittleness and excessive pressure drops across the filters. To mitigate this risk, relatively larger gel filters can be used. Gel filters suitable for flow rates up to 135 lpm are commercially available. In environments with unusually high particulate loading, such as busy subways, the filter magazine can hold extra filters, allowing more frequent automated filter change-out. Timing for this change-out can be motivated by a continuous measurement of the pressure drop across the filter. Desiccation of the gel filter due to desorption of water can be mitigated by controlling the temperature and humidity of the incoming air to a range suitable range for the gel filters. Softening of the gel filter due to adsorption of water vapor will be mitigated by avoiding high humidity conditions through heating/drying the incoming air. Where ambient conditions are both high temperature and high humidity, condensing techniques may be required to remove excess moisture. Thus, in at least one embodiment, the humidity control component will include the ability to remove moisture from ambient air entering the inlet/hydration tube, before such air reaches the gel filter.

Loss of viability of the collected biological particles will be minimized by controlling the incoming air humidity and temperature during collection. Storage at 4° C. should be suitable for maintaining viability of most targets for several days, if not several weeks. If it is determined that a single condition is not suitable for maintaining viability of all targets of interest, then the system can be expanded to sample onto separate filters in parallel, and then be stored in separate compartments maintained under different conditions. The flow conditioning tube can be shared by the filters, or each parallel filter can be serviced by an inlet tube whose temperature and humidity conditions are separately controlled.

As noted above, the controller used to implement the functions discussed above can be implemented as a hardware controller (such as an application-specific integrated circuit) or as a software-based controller (i.e., a computing device including a processor that executes machine instructions stored in a memory to carry out control functions). The following briefly discusses exemplary functions that can be implemented by such a controller. It should be understood that the following functions can be implemented in various permutations and combinations.

Where the sampler includes a plurality of collection surfaces, the controller can be used to control when the collection surface is replaced. Thus, one exemplary function is to move a fresh collection surface to a sampling position in response to the detection of a pressure drop across the collection surface. Another exemplary function is to move a fresh collection surface to a sampling position in response to the lapse of a predetermined interval of time. Another exemplary function is to move a fresh collection surface to a sampling position in response to determining that a predetermined mass of particulates has been collected. Still another exemplary function is to move a fresh collection surface to a sampling position in response to a user input.

As discussed above, exemplary samplers include sensors that provide data to the controller to be used to establish environmental conditions to maintain the viability of the collected biological particles. Thus, one exemplary function is to monitor the temperature of the ambient air, and to modify that temperature in the hydration tube. A related exemplary function is to monitor the humidity of the ambient air, and to modify that temperature in the hydration tube. The temperature and/or humidity of the ambient air can be measured outside of the housing, or as the air enters the inlet portion of the hydration tube. In embodiments including a fresh filter magazine, one exemplary function is to monitor a temperature in a volume defined by the fresh filter magazine, and to control that temperature to reduce microbial growth on collection substrates stored therein. A related exemplary function is to monitor the humidity in the volume defined by the fresh filter magazine, and to control that humidity to reduce microbial growth on collection substrates stored therein. In embodiments including a spent filter magazine, one exemplary function is to monitor a temperature in a volume defined by the spent filter magazine, and to control that temperature to reduce microbial growth on collection substrates stored therein. A related exemplary function is to monitor the humidity in the volume defined by the spent filter magazine, and to control that humidity to reduce microbial growth on collection substrates stored therein. In general, relatively lower temperatures will reduce microbial growth.

The terms about and approximately, as used above and in the claims that follow, should be understood to encompass a specified parameter, plus or minus 10%.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for collecting viable biological particles from ambient air, and maintaining the viability of the collected particles over an extended period of time, comprising the steps of:
(a) collecting a sample of particles from the ambient air using a particle collector; and
(b) maintaining temperature and humidity conditions within the particle collector at levels that are selected to maintain a viability of the collected biological particles.

2. The method of claim 1, wherein the step of maintaining temperature and humidity conditions within the particle collector at levels that are selected to maintain a viability of the collected biological particles comprises the steps of:
(a) controlling at least one of a temperature and a humidity of air entering the particle collector; and
(b) controlling at least one of a temperature and a humidity of a volume in which collected particles are stored within the particle collector.

3. The method of claim 1, wherein the step of maintaining temperature and humidity conditions within the particle collector at levels that are selected to maintain a viability of the collected biological particles comprises the step of controlling the humidity to achieve a humidity level of at least about 75%, but not greater than about 99%, (non-condensing).

4. The method of claim 1, wherein the step of maintaining temperature and humidity conditions within the particle collector at levels that are selected to maintain a viability of the collected biological particles comprises the step of controlling the temperature condition to achieve a temperature ranging from about 4 to about 30 degrees Celsius.

5. The method of claim 1, wherein the step of collecting the sample of particles from the ambient air comprises the steps of:
(a) filtering the ambient air to remove particles larger than a desired size; and
(b) directing the filtered ambient air through a temperature and humidity controlled volume toward a collection surface.

6. The method of claim 5, wherein the step of directing the filtered ambient air through the temperature and humidity controlled volume comprises the step of directing the filtered ambient air through a porous hydration tube.

7. The method of claim 5, further comprising the step of automatically replacing a spent collection surface with a fresh collection surface.

8. The method of claim 7, wherein the step of automatically replacing the spent collection surface with a fresh collection surface comprises the step of replacing the spent collection surface when at least one of the following conditions is satisfied:
(a) a first condition corresponding to a passage of a predetermined time interval;
(b) a second condition corresponding to a predetermined increase in a pressure drop across the collection surface; and
(c) a third condition corresponding to the collection of a predetermined mass of particles.

9. The method of claim 7, wherein the step of automatically replacing the spent collection surface with a fresh collection surface comprises the step of storing the spent collection surface in a magazine in which the temperature and humidity is controlled to enhance a viability of the collected biological particles.

10. The method of claim 5, further comprising the step of collecting a sample of the collected biological particles from the collection surface by dissolving the collection surface with a solvent to generate a liquid sample.

11. A viable biological particle sampler for collecting biological particles from ambient air, and maintaining a viability of the collected biological particles over an extended period of time, the viable biological particle sampler comprising:
(a) a housing;
(b) an air pump configured to introduce ambient air containing viable biological particles into the housing;
(c) a collection surface disposed within the housing upon which viable biological particles contained within the ambient air introduced into the housing are deposited;
(d) a temperature control component configured to maintain a temperature in at least one volume in the sampler at a level selected to maintain the viability of the collected biological particles; and
(e) a humidity control component configured to maintain a humidity level in at least one volume in the sampler at a level selected to maintain the viability of the collected biological particles.

12. The viable biological particle sampler of claim 11, further comprising:
(a) a size selecting component coupled to an inlet through which the ambient air is introduced into the housing, the size selecting component removing particles larger than a predefined size;
(b) an exhaust port for discharging the ambient air drawn into the housing by the air pump after the viable biological particles entrained in the ambient air are deposited on the collection surface;
(c) a power supply; and
(d) a controller logically coupled to the air pump, the power supply, the humidity control component, and the temperature control component.

13. The viable biological particle sampler of claim 11, further comprising a porous hydration tube in fluid communication with the inlet, the collection surface, and the humidity control component.

14. The viable biological particle sampler of claim 11, wherein the humidity control component comprises a hydration pump and a hydration reservoir.

15. The viable biological particle sampler of claim 11, wherein the hydration pump is configured to deliver water from the hydration reservoir to walls of the porous hydration tube, thereby increasing the humidity inside of the hydration tube.

16. The viable biological particle sampler of claim 14, wherein the humidity control component further comprises a scavenging component configured to scavenge moisture from the ambient air introduced into the housing after the biological particles are deposited onto the collection surface and before the ambient air is discharged from the housing.

17. The viable biological particle sampler of claim 11, wherein the humidity control component further comprises a sensor configured to determine a humidity level in the at least one volume, such that when the humidity level drops below a predetermined value, the humidity control component responds by increasing the humidity level.

18. The viable biological particle sampler of claim 11, wherein the temperature control component further comprises a sensor configured to determine a temperature in the at least one volume, such that when the temperature drops below a predetermined value, the temperature control component responds by increasing the temperature.

19. The viable biological particle sampler of claim 11, further comprising:
(a) a first magazine configured to store a plurality of fresh collection surfaces;
(b) a second magazine configured to store a plurality of spent collection surfaces; and
(c) a collection surface transfer component configured to:
(i) move a fresh collection surface from the first magazine to a sampling position where viable biological particles are deposited on that collection surface; and
(ii) move a spent collection surface from the sampling position to the second magazine.

20. The viable biological particle sampler of claim 19, wherein at least one of the magazines is refrigerated.

21. The viable biological particle sampler of claim 19, wherein the humidity control component and the temperature control component manage temperature and humidity conditions in the second magazine to levels selected to maintain the viability of biological particles deposited on spent collection surfaces.

22. The viable biological particle sampler of claim 19, wherein the collection surface transfer component comprises a carousel and a prime mover.

23. The viable biological particle sampler of claim 11, further comprising a liquid sample collection component configured to acquire a liquid sample containing particles deposited onto the collection surface by dissolving the collection surface.

24. A viable biological particle sampler for collecting biological particles from ambient air, and maintaining a viability of the collected biological particles over an extended period of time, the viable biological particle sampler comprising:
(a) a housing;
(b) an air pump configured to introduce ambient air containing viable biological particles into the housing;
(c) a sampling position disposed in fluid communication with the ambient air introduced into the housing are deposited;
(d) a first magazine configured to store a plurality of fresh collection surfaces;
(e) a second magazine configured to store a plurality of spent collection surfaces;
(f) a collection surface transfer component configured to:
(i) move a fresh collection surface the first magazine to the sampling position so that viable biological particles are deposited on that collection surface; and
(ii) move a spent collection surface from the sampling position to the second magazine;
(g) a temperature control component configured to maintain a temperature in at least one volume in the sampler at a level selected to maintain the viability of collected biological particles; and
(h) a humidity control component configured to maintain a humidity level in at least one volume in the sampler at a level selected to maintain the viability of collected biological particles; wherein the at least one volume comprises at least one volume selected from a group of volumes consisting of:
(i) a first volume defined by the housing;
(ii) a second volume defined by the second magazine for storing the spent collection surfaces; and
(iii) a third volume defined by a fluid conduit coupling a housing inlet by which ambient air is introduced into the housing and the sampling position in fluid communication.

25. The viable biological particle sampler of claim 24, further comprising:
(a) a size selecting component coupled to the inlet, the size selecting component removing particles larger than a predefined size;
(b) an exhaust port for discharging the ambient air drawn into the housing by the air pump after the viable biological particles entrained in the ambient air are deposited on a collection surface disposed at the sampling position;
(c) a power supply; and
(d) a controller logically coupled to the air pump, the power supply, the humidity control component, the temperature control component, and the collection surface transfer component.

26. The viable biological particle sampler of claim 24, further comprising an additional fluid conduit coupling the inlet to an additional sampling position, such that the temperature and humidity control components establish different temperature and humidity conditions in the fluid conduit and the additional fluid conduit.
